(19) United States
(12) Patent Application Publication
(10) Pub. No.: US 2006/0147559 A1
(43) Pub. Date: Jul. 6, 2006

(54) Methods for Altering the Mineral Content of Foods

(75) Inventors: Gerald L. Maurer, Cincinnati, OH (US); J. David Genders, Elma, NY (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 11/306,648

(22) Filed: Jan. 5, 2006

Related U.S. Application Data
(60) Provisional application No. 60/593,330, filed on Jan. 6, 2005.

(51) Int. Cl. A61K 36/18

(52) U.S. Cl. 424/725

(57) ABSTRACT

The present invention involves removing of particular ions, particularly ionic potassium, from juices via an electrodialysis system and replacing the removed ions with other nutrients or beneficial ions, such as, calcium.

### CROSS-REFERENCE TO RELATED APPLICATION

This application for a patent claims priority to U.S. Provisional Patent Application No. 60/593,330 as filed Jan. 6, 2005.

### BACKGROUND

The various exemplary embodiments of the present invention relate to the altering of mineral content of foods, more particularly, the various exemplary embodiments of the present invention relate to the removal or replacement of potassium in juice drinks.

The careful control of ingested potassium ions (K⁺) is of vital importance to particular patients, especially those suffering from end-stage renal disease (ESRD). It is of vital importance due to such patients' inability to maintain electrolyte homeostasis, a function typically performed by a normal healthy kidney.

Potassium can be especially problematic for ESRD patients because potassium, even in relatively minor amounts, can reach dangerous and/or lethal concentrations. This is because high potassium levels are known to interfere with cardiac muscle contractility, thereby leading to stoppage of the heart muscle. Additionally, persons suffering from diabetes often experience impaired kidney function raising the potential for hyperkalemia. This is especially true when patients, who are likewise at risk of hyperkalemia, are taking an angiotensin converting enzyme (ACE) inhibitor to treat hypertension and/or congestive heart failure.

Significant concentrations of potassium are found, for example, in particular fruit and vegetable juices. If not for the significant concentrations of potassium, such juices would be useful for ingestion of nutrients as well as providing drinking pleasure and enjoyment to ESRD patients and others using K⁺-sparing medications.

For example, orange juice, the most popular juice drink in the United States, has about 1200 to about 1800 milligrams (about 30 to about 46 mEq) of K⁺ per liter juice. In contrast, the normal concentration of K⁺ in human blood ranges from about 3.5 mEq to about 5.5 mEq per liter.

A concentration of K⁺ greater than 5.5 mEq per liter in the body is known as hyperkalemia, a potentially life-threatening condition. As should be readily appreciated, several glasses of orange juice would quickly increase the concentration of K⁺ in the body toward and possibly above a toxic range, especially if it cannot be properly controlled or expelled from the body.

Although the K⁺ in juices could be life-threatening to ESRD patients, the same juices, especially, for example, orange juice, comprises many important nutrients for physiological health. For example, orange juice includes vitamin C (ascorbic acid), as well as numerous other compounds favorable to human nutrition. In addition to the nutritional benefits, fruit and vegetable juices are refreshing foods having pleasant tastes and textures that would add to the quality of life of ESRD patients if the high concentration of K⁺ in the juice could be decreased to a level of non-toxicity.

Prior attempts at creating low concentration of K⁺ juices revolve around the use of cation-exchange resins. Such cation-exchanges are well-known in the art to remove cations or anions from aqueous mixtures, and can remove about 90% of K⁺ from the juice. However, such cation-exchange resins are highly dependent upon the nature of the actual resin used.

A problem exists in using such cation-exchange resins to remove K⁺ from juices. In particular, the cation-exchange resins essentially remove not only a majority of the K⁺ from the juice, but also many of the other cations in the juice. Other cations may include, for example, calcium (Ca²⁺) and other essential nutrients, flavor producing substances, and the like. As such cations are removed from the juice, they are replaced typically with a close to equal amount of hydrogen ions (H⁺), thereby leading to a highly reduced pH of the final juice product.

Typical cation-exchange resins can be regenerated and reused multiple times, but the initial cost can be expensive, upwards of about $200 per kilogram of resin. Multiple hundreds of kilograms of resin and associated mixing tanks, columns and regeneration chemicals combine to create a process that is highly expensive to establish, operate and maintain.

In addition to expensive costs, cation-exchange resins are not efficient. Batch-operated and column-operated ion exchange processes require an equilibrium time to allow the ions to actually exchange from the aqueous mixture and the resin. This equilibrium is dependent, for example, upon the ions, the nature of the aqueous mixture, viscosity, amount and presence of suspended solids, and the amount of resin employed.

For example, pulp from certain fruits and vegetables, such as, for example, oranges, tomatoes and prunes, increases the viscosity, thereby decreasing the exchange efficiency and fouling the resin. This interrupts the effective contact between the resin and the bulk of the aqueous mixture, and results in inefficient ion exchange and a high loss of juice components in the form of pulp. Such lost juice components are valuable for texture-imparting organoleptically favorable properties.

Further, although the ion-exchange resins are reusable, the resins still have a finite regenerative life such that they must be replaced periodically. These resins are also limited by the possible loading with cations, tiny fractions of cation weight compared to resin weight. Thousands of liters per day of K⁺ depleted juice must be prepared to meet the current demand.

Thus, what is desired is a means for decreasing the concentration of K⁺, but not other nutrients and valued components, in juices via a cost-effective, yet commercially scalable manner.

### SUMMARY

The various exemplary embodiments of the present invention include a process for substantially removing one or more predetermined ions from an aqueous mixture. The process comprises passing the aqueous mixture through a system, wherein the system comprises an ion-exchange membrane specifically selected to substantially remove ionic potassium. Next, a potential field is applied to the system, and the ionic potassium substantially removed is substituted with one or more predetermined ions, such that a resulting aqueous mixture comprises about 200 mg/L or less of ionic potassium.

The various exemplary embodiments of the present invention further comprise a process for substantially removing one or more predetermined monovalent ions from an aqueous mixture. The process comprises passing the aqueous mixture through a system, wherein the system comprises an ion-exchange membrane specifically selected to substantially remove monovalent ions. Next a potential field is applied to the system, and the one or more predetermined monovalent ions substantially removed are substituted with one or more predetermined multivalent ions, such that a resulting aqueous mixture comprises about 200 mg/L or less of the monovalent ions.

Additionally, the various exemplary embodiments of the present invention comprises a fruit or vegetable juice having about 200 mg/L or less of ionic potassium after being passed through a system, wherein the system comprises an ion-exchange membrane specifically selected to substantially remove monovalent ions and to which a potential field is applied.

### DETAILED DESCRIPTION

Various exemplary embodiments of the present invention comprise a process of substantially removing K⁺ from juices via specifically configured electrodialysis (ED) cells and associated equipment. It has been found that ED removes K⁺ very efficiently and rather specifically, unlike ion-exchange resins. Further, ED is very fast and efficient.

Electrodialysis essentially is a membrane process in which a flowing aqueous mixture contacts one or more ion-exchange membranes under an applied potential field.

Further, the essentially one-time costs of ED equipment can be amortized rather quickly because of the very high throughput of product and minimal replacement of the ion-specific membranes employed in an ED process.

Additionally, known ion-exchange resin methods include a built-in downtime for regeneration of the associated resin. The downtime for ED equipment is comparatively a fraction of the ion-exchange resin downtime, and thereby results in greater production time with the ED process.

Electrodialysis is advantageous in that it can specifically target substantial removal of one or more particular ions, such as, for example, K⁺, from an aqueous mixture. While specifically targeting removal of one or more ions, the other cations and natural species of the aqueous mixture remain in the aqueous mixture. In particular, in the exemplary embodiments of the present invention, monovalent ions are selectively removed from an aqueous mixture and multivalent ions remain in the aqueous mixture. Further, the amount of multivalent ions may be increased in the aqueous mixture.

In the various exemplary embodiments of the present invention, in addition to substantially removing particular ions from an aqueous mixture, other particular ions can simultaneously be introduced to the aqueous mixture, thereby essentially replacing the ions substantially removed from the aqueous mixture.

For example, it has been shown via the various exemplary embodiments according to the present invention that K⁺ can be substantially removed from apple juice, and the K⁺ removed can be replaced by calcium ions (Ca²⁺).

Electrodialysis is a membrane process in which ions are transported through ion exchange membranes under the influence of a potential field. When the fruit juice is pumped through the feed compartment of a membrane stack and an electric potential is applied between an anode and cathode, positively charged cations migrate toward the cathode and negatively charged anions migrate toward the anode. The cations pass through the negatively charged cation exchange membranes but are largely rejected by the positively charged anion exchange membranes, if used. In addition, monovalent selective cation exchange membranes can be used which preferentially allows monovalent cations to pass into or out of selected compartments and reject divalent and larger cation species. Likewise, the negatively charged anions pass through the anion exchange membranes but are rejected by the cation exchange membranes. The overall result is a decrease in the K⁺ concentration of the juice stream and an increase in the concentrate stream when both anion and cation exchange membranes are used, or a loss of K⁺ and replacement with Ca²⁺ in the juice stream when a combination of cation exchange membranes is used.

In the various exemplary embodiments of the present invention, any commercially available electrodialysis apparatus using an ion-permselective membrane can be employed.

Throughout the processing of the aqueous mixture, the pH, inlet pressure and conductivity should be continuously monitored to ensure consistency.

In an exemplary embodiment, the ED run is carried out in an ESC ED-1 electrolytic stack. The stack comprises a platinized titanium anode, 316 stainless steel cathode and one of a Neosepta AMX anion and CMX cation exchange membranes combination or a Neosepta CMS and CMX cation exchange membranes combination. Neosepta CMS membranes are selective for monovalent cations. Gaskets are 1/16 inch thick and are comprised of EPDM and the spacers are comprised of polypropylene. There are 5 ED membrane pairs, each with an operating surface area of about 0.011 m². The feed compartment comprises a 2 L glass reservoir and a March AC-3C-MD centrifugal circulating pump.

In the exemplary embodiment, a concentrate loop comprises a 1 L glass reservoir and a March AC-3C-MD centrifugal circulating pump. The inlet pressure, pH and conductivity of this solution is monitored throughout the run. The starting concentrate solution may comprise water or CaCl₂ solution having a concentration of about 0.13M to about 0.5M.

An electrode rise loop of the system according to an exemplary embodiment comprises a 1 L glass reservoir and a March AC-3C-MD centrifugal circulating pump. The electrode rinse solution may comprise of 0.2M Na₂SO₄. The electrode rinse solution may be split into two streams before entering the anode and cathode compartments. The solutions exiting the compartments may be recombined in the main reservoir to maintain pH neutrality in the rinse solution.

Power may be supplied by a DC power supply, such as, for example, a Hewlett Packard 6010A DC power supply.

In various exemplary embodiments, a current density of less than about 10 mA/cm² and greater than about 1.0 mA/cm² is desired better ensure adequate removal of K⁺ from an aqueous solution.

Aqueous mixtures, such as, for example, juices having pulp, can be optionally filtered prior to processing according to the various exemplary embodiments of the present invention.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. A process for substantially removing one or more predetermined ions from an aqueous mixture, comprising:
passing the aqueous mixture through a system, wherein the system comprises an ion-exchange membrane specifically selected to substantially remove ionic potassium;
applying a potential field to the system; and
substituting the ionic potassium with one or more predetermined ions,
wherein a resulting aqueous mixture comprises about 200 mg/L or less of ionic potassium.

2. The process according to claim 1, wherein the aqueous mixture is selected from the group consisting of fruit juice, vegetable juice, and a combination thereof.

3. The process according to claim 1, wherein the resulting mixture comprises about 10% or less of ionic potassium of the ionic potassium from the aqueous mixture.

4. The process according to claim 1, wherein the system includes electrodialysis cells.

5. The process according to claim 1, wherein the aqueous mixture is filtered prior to passing through the system.

6. The process according to claim 1, wherein the one or more predetermined ions is ionic calcium.

7. A process for substantially removing one or more predetermined monovalent ions from an aqueous mixture, comprising:
passing the aqueous mixture through a system, wherein the system comprises an ion-exchange membrane specifically selected to substantially remove monovalent ions;
applying a potential field to the system; and
substituting the one or more predetermined monovalent ions substantially removed with one or more predetermined multivalent ions,
wherein a resulting aqueous mixture comprises about 200 mg/L or less of the monovalent ions.

8. The process according to claim 7, wherein the monovalent ions are cationic.

9. The process according to claim 7, wherein the multivalent ions are cationic.

10. The process according to claim 7, wherein the monovalent ions comprise ionic potassium.

11. The process according to claim 7, wherein the multivalent ions comprise ionic calcium.

12. A fruit or vegetable juice having about 200 mg/L or less of ionic potassium after being passed through a system, wherein the system comprises an ion-exchange membrane specifically selected to substantially remove monovalent ions and to which a potential field is applied.
